(19) United States
(12) Patent Application Publication
(10) Pub. No.: US 2023/0172907 A1
(43) Pub. Date: Jun. 8, 2023

(54) SARS-COV-2 INHIBITORS

(71) Applicant: KING FAISAL UNIVERSITY, Hofouf (SA)

(72) Inventors: MAHMOUD KANDEEL, Hofouf (SA); HYUNG-JOO KWON, Hofouf (SA)

(21) Appl. No.: 17/831,560

(22) Filed: Jun. 3, 2022

Related U.S. Application Data
(60) Provisional application No. 63/286,143, filed on Dec. 6, 2021.

(51) Int. Cl. A61K 31/409; A61K 45/06; A61P 31/14

(52) U.S. Cl. CPC A61K 31/409; A61K 45/06; A61P 31/14

(57) ABSTRACT

SARS-CoV-2 inhibitors include pharmaceutical compounds capable of binding to the active site of SARS-CoV-2 Mpro. The SARS-CoV-2 inhibitors may be used in pharmaceuticals to prevent and/or treat SARS-CoV-2 infection. The pharmaceuticals may be formulated to comprise at least one SARS-CoV-2 inhibitor and a carrier, or they may include at least one SARS-CoV-2 inhibitor and a further pharmaceutical compound known to be effective against SARS-CoV-2.

### CROSS-REFERENCE TO RELATED APPLICATION

This application claims the benefit of U.S. Provisional Pat. Application No. 63/286,143, filed on Dec. 6, 2021.

### BACKGROUND

### 1. Field

The disclosure of the present patent application relates to anti-RNA virus compositions, and particularly, to SARS-CoV-2 inhibitors.

### 2. Description of the Related Art

The recent outbreak of SARS-CoV-2 has been declared a pandemic by WHO. The disease COVID-19 causes a range of symptoms, from mild respiratory symptoms to severe respiratory distress associated with sepsis, multi-organ dysfunction, and death (Zaim et al., “COVID-19 and multi-organ response,” Current Problems in Cardiology: 10061 8, 2020). The current alarming situation necessitates the rapid reallocation or repurposing of previously known drugs or chemical compounds for the use in treating COVID-19.

Approximately seven human coronaviruses (HCoV) have been identified. Four CoVs were identified as causative agents for mild respiratory symptoms and the common cold, including HCoV-229E, HCoV-NL63, HCoV-OC43, and HCoV-HKU1 (Zeng et al., “Epidemiology and clinical characteristics of human coronaviruses OC43, 229E, NL63, and HKU1: a study of hospitalized children with acute respiratory tract infections in Guangzhou, China,” European Journal of Clinical Microbiology & Infectious Diseases, 37: pp. 363-369, 2018). However, more recently, severe symptoms and fatal outcomes have been caused by three other epidemic viruses, including SARS-CoV, MERS-CoV, and SARS-CoV-2. Coronaviruses express their nonstructural protein in the form of a large protein called polyprotein AB. This polyprotein must be processed by the host as well as the viral encoded proteases to release approximately 16 nonstructural proteins (“NSPs”). Two viral proteases share in the digestion of polyprotein AB: the main protease, called 3-C-like protease (“Mpro”), and a papain-like protease (“PLpro”) (Hilgenfeld, R., “From SARS to MERS: crystallographic studies on coronaviral proteases enable antiviral drug design,” The FEBS Journal, 281: 4085-4096, 2014). Both PLpro and Mpro have been important targets for drug discovery against SARS CoV, MERS CoV, and SARS CoV-2 (Kandeel et al., “Molecular Dynamics and Inhibition of MERS CoV Papain-like Protease by Small Molecule Imidazole and Aminopurine Derivatives,” Letters in Drug Design & Discovery, 16: 584-591, 2019; Li et al., “Molecular Characteristics, Functions, and Related Pathogenicity of MERS-CoV Proteins,” Engineering, 2019; Pillaiyar et al., “Recent discovery and development of inhibitors targeting coronaviruses,” Drug Discovery Today, 2020; Zumla et al., “Coronaviruses-drug discovery and therapeutic options,” Nature reviews Drug Discovery, 15: 327, 2016).

Recently, computational details regarding targeting the Mpro and PLpro were provided with a dataset of FDA approved drugs (Kandeel et al., “Repurposing of FDA-approved antivirals, antibiotics, anthelmintics, antioxidants, and cell protectives against SARS-CoV-2 papain-like protease,” Journal of Biomolecular Structure and Dynamics: pp. 1-8, 2021; Kandeel & Al-Nazawi, “Virtual screening and repurposing of FDA approved drugs against COVD-19 main protease,” Life Sciences: 117627, 2020).

While a large number of drug repurposing studies have been conducted, the need for more and better therapeutic options for treating SARS-CoV-2 remains. Thus, SARS-CoV-2 inhibitors solving the aforementioned problems are desired.

### SUMMARY

SARS-CoV-2 inhibitors include a set of pharmaceutical compounds capable of binding to the SARS-CoV-2 Mpro protein. The SARS-CoV-2 inhibitors can inhibit SARS-CoV-2 infection in cells and may be used to prevent and/or treat SARS-CoV-2 infection.

An embodiment of the present subject matter is directed to methods of inhibiting SARS-CoV-2 infection, preventing SARS-CoV-2 transmission, and/or treating a SARS-CoV-2 infection, including administering to a subject in need thereof a therapeutically effective amount of a pharmaceutical composition including a SARS-CoV-2 inhibitor according to the present subject matter.

These and other features of the present subject matter will become readily apparent upon further review of the following specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A: depicts a 3D model of the docking site ligand interactions with Mpro following the XP docking protocol.

FIG. 1B: depicts a 3D model of the docking site ligand interactions with Mpro with a surface representation (blue) of birinapant in the active site of Mpro.

FIG. 1C: depicts a 3D model of docking site ligand interactions with Mpro with the binding site of residues of atazanavir D.

FIG. 1D: depicts a 3D model of docking site ligand interactions with Mpro with the binding site residues of birinapant.

FIG. 1E: depicts a schematic diagram of site ligand interactions with Mpro with the ligand interactions of atazanavir F.

FIG. 1F: depicts a schematic diagram of docking site ligand interactions with Mpro with the ligand interactions of birinapant.

FIGS. 2A-2C: depict RMSD plots of the top fourteen compounds after MDS for 20 ns. Lopinavir was used for reference. Apo structure is Mpro without any ligands.

FIGS. 3A-3C: depict RMSD plots of the top four compounds, alpha-mangostin, atazanavir, birinapant, and lopinavir, after MDs for 100 ns.

FIG. 4: depict a RMSF plot of the top four compounds after MDs for 100 ns.

FIG. 5: depict the radius of gyration of the top four compounds after MDs for 100 ns.

FIG. 6: depict the hydrogen bond length of the top four compounds after MDs for 100 ns.

FIG. 7: depict a graph of the effect of birinapant and atazanavir on the replication of SARS-CoV-2.

Similar reference characters denote corresponding features consistently throughout the attached drawings.

### DETAILED DESCRIPTION OF THE PREFERRED EMBODIMENTS

A method of treating or preventing SARS CoV-2 infection of a cell can include administering at least one SARS-CoV-2 inhibitor to a subject in need thereof. In an embodiment, the SARS CoV-2 inhibitor is birinapant. In an embodiment, the SARS-CoV-2 inhibitor is a pharmaceutical compound capable of binding to the SARS-CoV-2 Mpro protein. The SARS-CoV-2 inhibitors are capable of inhibition of SARS-CoV-2 infection in cells and may be used to prevent and/or treat SARS-CoV-2 infection.

Throughout this application, the term “about” may be used to indicate that a value includes the standard deviation of error for the composition, device or method being employed to determine the value.

The use of the term “or” in the specification and claim(s) is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps. In certain cases, the term “comprising” may be replaced with “consisting essentially of” or “consisting of.”

The use of the word “a” or “an” when used herein in conjunction with the term “comprising” in the claims and/or the specification may mean “one,” but it is also consistent with the meaning of “one or more,” “at least one,” and “one or more than one.”

The phrase “pharmaceutically acceptable,” as used herein, refers to molecular entities and compositions that do not produce an allergic or similar untoward reaction when administered to a human.

The term “subject,” as used herein, means a mammal, including but not limited to a human being.

As used herein, the term “providing” an agent is used to include “administering” the agent to a subject.

As used herein, a “carrier” includes any and all solvents, dispersion media, vehicles, coatings, diluents, isotonic and absorption delaying agents, buffers, carrier solutions, suspensions, colloids, excipients, and the like.

As used herein, “birinapant” refers to a SMAC mimetic antagonist having the chemical formula C₄₂H₅₆F₂N₈O₆, and currently being investigated for its ability to induce apoptosis in latent HIV-1 infected cells. Birinapant is also referred to as 1260251-31-7, TL32711, or TL-32711.

An embodiment of the present subject matter is directed to a method of making a pharmaceutical composition including mixing one or more of the SARS-CoV-2 inhibitors with a pharmaceutically acceptable carrier. For example, the method of making a pharmaceutical composition can include mixing a SARS-CoV-2 inhibitor under sterile conditions with a pharmaceutically acceptable carrier with preservatives, buffers, and/or propellants to create the pharmaceutical composition.

An embodiment of the present subject matter is directed to use of a pharmaceutical composition including one or more of the SARS-CoV-2 inhibitors. To prepare the pharmaceutical composition, one or more of the SARS-CoV-2 inhibitors, as the active ingredient, are intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. One or more of the SARS-CoV-2 inhibitors can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of a SARS-CoV-2 inhibitor or an amount effective to treat a disease, such as a coronavirus infection, may be determined initially from the Examples described herein and adjusted for specific targeted diseases using routine methods.

The SARS-CoV-2 inhibitors can be administered to a subject in need thereof. In an embodiment, the SARS-CoV-2 inhibitors can be administered to a subject in need thereof to inhibit SARS-CoV-2 infection, prevent SARS-CoV-2 transmission, and/or treat a SARS-CoV-2 infection.

An embodiment of the present subject matter is directed to a method of inhibiting SARS-CoV-2 infection, preventing SARS-CoV-2 transmission, and/or treating a SARS-CoV-2 infection, comprising administering to a subject in need thereof a therapeutically effective amount of the pharmaceutical composition according to the present subject matter.

The SARS-CoV-2 inhibitors or pharmaceutical compositions thereof can be administered to a subject by any suitable route. For example, the compositions can be administered nasally, rectally, intracisternally, intraperitoneally, transdermally (as by powders, ointments, or drops), and/or parenterally. As used herein, “parenteral” administration refers to modes of administration other than through the gastrointestinal tract, which includes intravenous, intramuscular, intraperitoneal, intrasternal, intramammary, intraocular, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

Accordingly, the route of administration can include intranasal administration, oral administration, inhalation administration, subcutaneous administration, transdermal administration, intradermal administration, intra-arterial administration with or without occlusion, intracranial administration, intraventricular administration, intravenous administration, buccal administration, intraperitoneal administration, intraocular administration, intramuscular administration, implantation administration, topical administration, intratumor administration, and/or central venous administration.

In this study, a large library of virus protein-specific compounds was selected. Initial docking showed interesting docking scores and favorable profiles of structure stability and binding energy. The four drugs selected for the final comprehensive (100-ns MD) simulations were alpha-mangostin, atazanavir, birinapant and lopinavir.

Lopinavir is a well-known protease inhibitor with high efficiency against the HIV-1 protease. In addition, the drug was effective against SARS-CoV and MERS-CoV and improved the health markers in SARS-CoV-2-infected patients. Atazanavir is another HIV-1 protease inhibitor with the advantage of oral administration in combination with other antiretroviral drugs. Birinapant is an apoptosis inhibitor and has approved efficiency in controlling viral hepatitis in combination with other antiviral drugs. In comparison with lopinavir, both atazanavir and birinapant showed an improved XP-docking score, higher binding energy and a lower structural root-mean-square deviation (RMSD) during 100-ns MD simulations. Therefore, based on the clinical efficiency of lopinavir against SARS-CoV-2, the drugs atazanavir and birinapant can be expected to perform with similar or improved efficacy comparable to that of ritonavir.

After a comprehensive study involving virtual screening, docking, and MD simulations of a unique set of antiviral agents, two highly potent Mpro-binding drugs, birinapant and atazanavir, showed promise. These drugs had improved energetic and structural stability profiles that were comparable to or higher than those produced by the classic antiviral protease inhibitor ritonavir. Birinapant was found to inhibit SARS-CoV-2 replication with promising inhibition in the low micromolar range. The observed antiviral action of birinapant is derived by multiple strong binding with Mpro and Spike’s RBD.

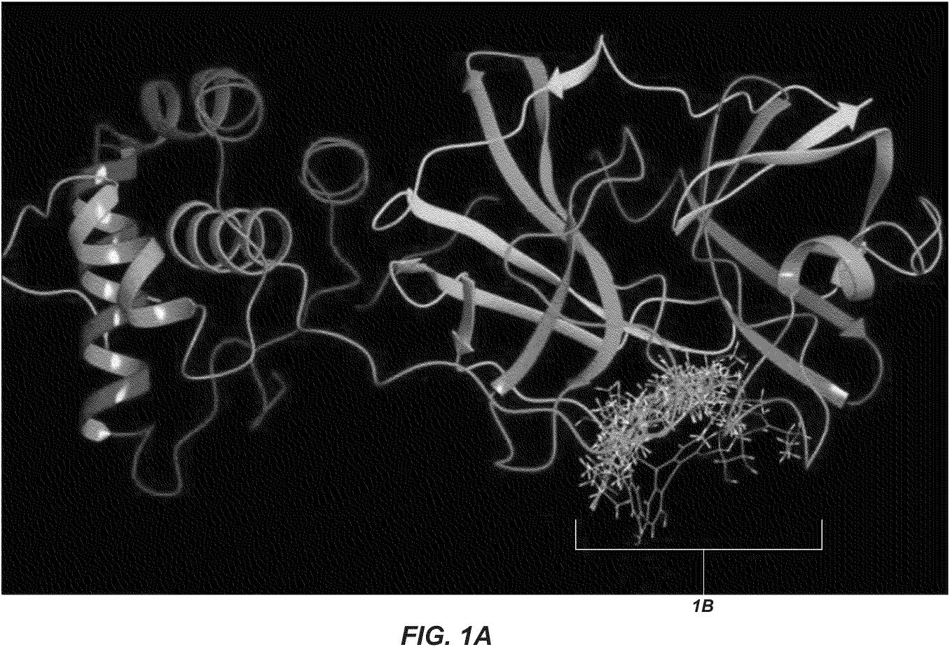

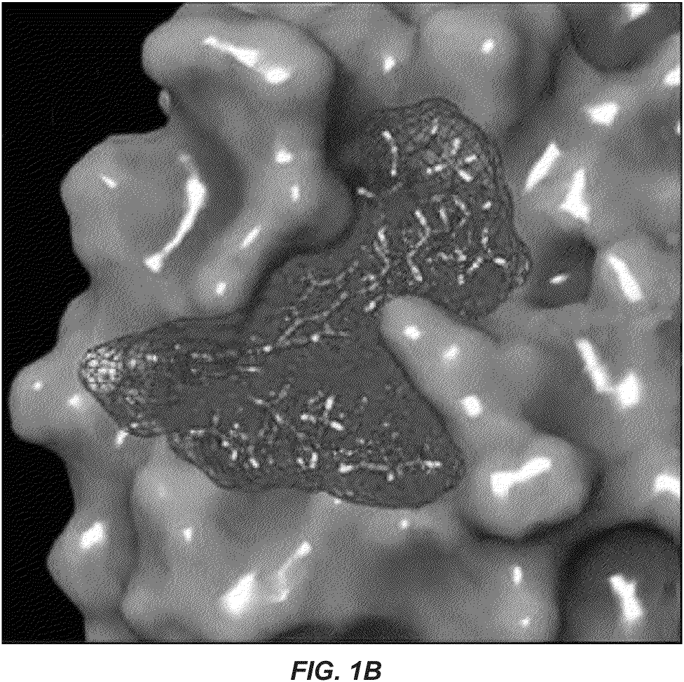

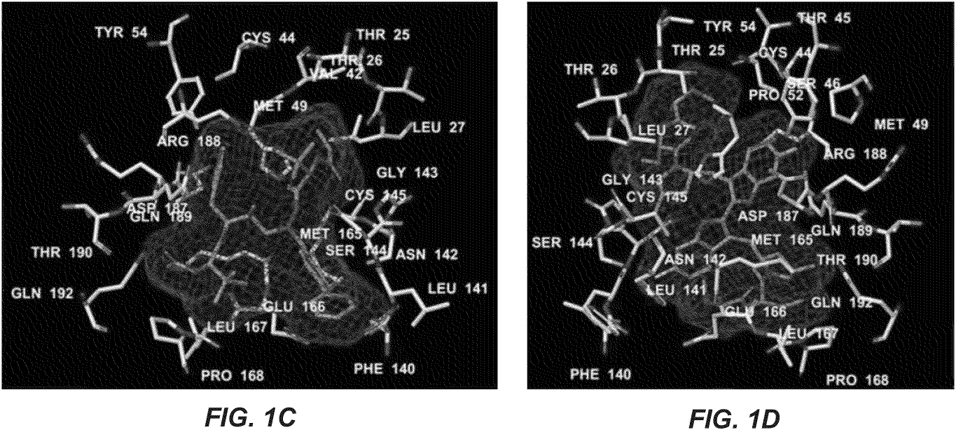

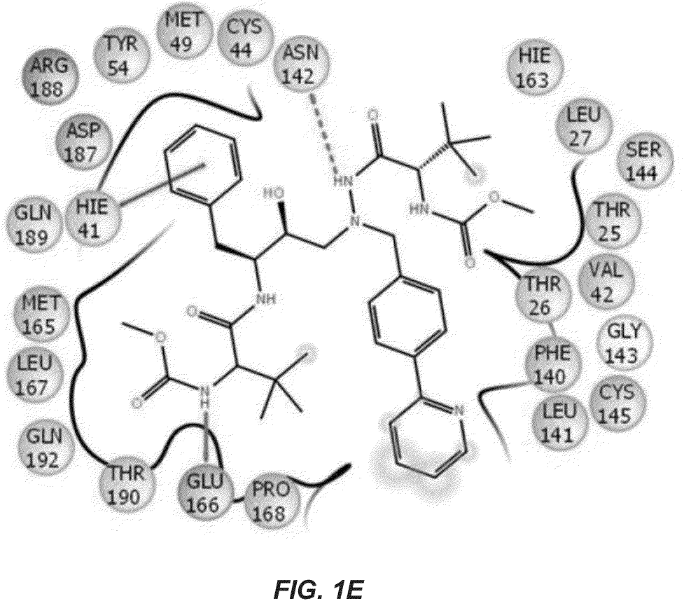

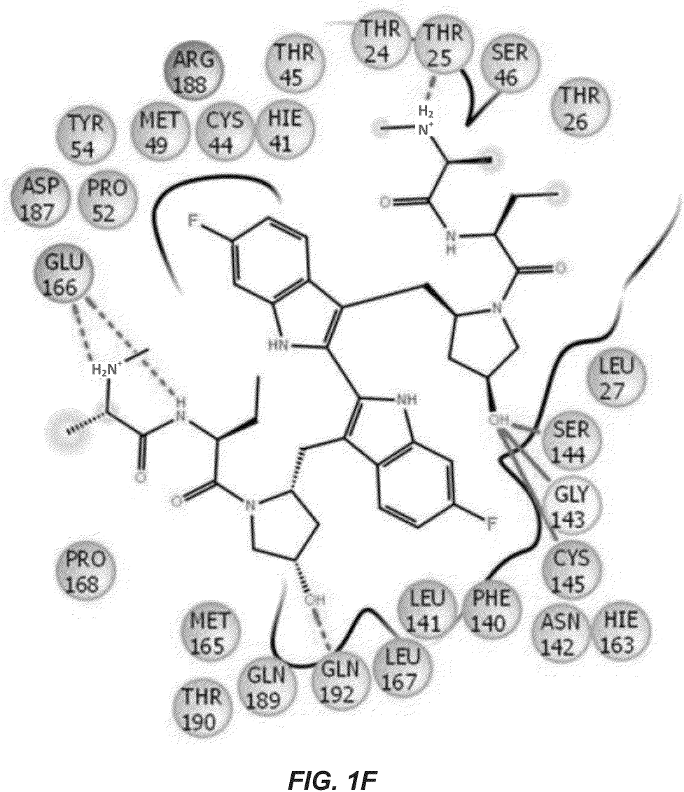

The strong binding profiles of atazanavir and birinapant are supported by hydrophobic interactions. In addition, atazanavir formed two hydrogen bonds with ASN142 and GLU166, and birinapant formed four hydrogen bonds with THR25, GLU166, and GLN192 and a tridentate bond with GLY143, SER144 and CYS145 (FIG. 1E and F).

In the second rank, following atazanavir and birinapant, alpha-mangostin and cobicistat showed quite high binding energy but had less structural stability owing to higher RMSD values. Cobicistat is a cytochrome enzyme inhibitor used to increase the systemic availability of other antiviral drugs, such as atazanavir.

After screening investigations, the top two compounds were used in antiviral assays. Atazanavir had no antiviral effects. In contrast, birinapant has antiviral properties at low micromolar concentrations. Birinapant’s anti-SARS-CoV-2 activity supports its usage as an anti-COVID-19 medication. Combining birinapant with at least one anti-viral agent may result in considerable SARS-CoV-2 virus particle elimination.

After a comprehensive study involving virtual screening, docking, and MD simulations of a unique set of antiviral agents, two highly potent Mpro-binding drugs, birinapant and atazanavir, showed promise. These drugs had improved energetic and structural stability profiles that were comparable to or higher than those produced by the classic antiviral protease inhibitor ritonavir. Birinapant was found to inhibit SARS-CoV-2 replication with promising inhibition in the low micromolar range. The observed antiviral action of birinapant is derived from multiple strong binding with Mpro and Spike’s RBD.

The following examples illustrate the present subject matter.

### EXAMPLES

The present inventors appreciate the support that the Ministry of Health of Saudi Arabia provided for the research and experiments described herein.

### Example 1

### Construction of Drugs and Compounds Dataset, Ligand Preparation, Virtual Screening, Simulation, and Analysis

A total of 37060 compounds dataset was constructed comprising SARS CoV-2 main protease targeted library, compound obtained from 2D fingerprint from therapeutically relevant antiviral assays, combined ligand and structure-based approaches of inhibitors of viral proteins (See Table 1). All compounds were imported to Ligprep software, desalted and 3D optimized using OPLS2005 force field at physiological pH.

TABLE 1

The Compound Dataset

| Library Name | Company | No. Cmpds | Method of Selection |
|---|---|---|---|
| Main Protease Targeted Library | Life Chemicals (Niagara-on-the-Lake, ON, Canada) | 2300 | Glide by Schrödinger, SP mode was used to search Life chemicals HTS collection, by using the main protease of SARS CoV-2 in complex with an inhibitor N3. |
| Antiviral Library by 2D Simulation | Life Chemicals | 19244 | Antiviral Screening Compounds Library was designed with 2D fingerprint similarity search against the 41,514 biologically active compounds from therapeutically relevant viral assays from different virus species. |
| Antiviral Library by Combined Ligand0base & Structure-based Approaches Antiviral Library | Life Chemicals | 3500 | Antiviral protein targets were collected from the RCSB PDB. The reference antivirals were collected from ChEMBLdb and clustered according to the target. The top compounds were docked into the target protein and ranked. |
| | Asinex (Winston-Salem, NC, USA) | 6827 | Small molecules and macrocycles with antiviral activity. Specific designs include a-helix mimetics, glycomimetic, diverse synthetic macrocyles, and tri/tetra-substituted scaffolds. |
| Enamine Antiviral Library | Enamine (Monmouth Jct., NJ, USA) | 4842 | Nucleoside-like antiviral agents or Nucleoside mimetics from screening collection. The compounds contain natural-like moieties and diverse heterocycles as bioisosters of nucleosides. |
| Antiviral Compound Library | Selleck (Houston, TX, USA) | 347 | Collection of antiviral compounds. |
| | Total No. Compounds | 37,060 | |

The structures of Mpro (PDB ID 61u7) and RBD (PDB ID 7c8d) were retrieved from the protein data bank. The protein preparation wizard in Maestro software package (Schrodinger LLC, NY, USA) was used to process and optimize the protein structure. Water and other nonspecific molecules were removed, the protein was protonated to add polar hydrogens, the structure was optimized at cellular pH conditions, and the structure energy was minimized using OPLS2005 force field. The prepared structure was used in all docking and molecular dynamics calculations in this study.

Docking of all compounds was done by Schrodinger glide docking module. Two-step docking runs were carried out. Initially, the compounds were docked by the standard precision docking protocol (SP docking). Compounds with docking score of -8.00 or higher were retrieved and subjected to extra precision (XP-docking). The docking grid was generated by using the co-crystalized ligand as the center for docking box of 20 Å size. The obtained results were ranked according to the obtained docking scores.

Molecular dynamics (MD) simulations were carried out using Groningen Machine for Chemical Simulations (GROMACS) 5.1.4. (Abraham et al. 2015; Van Der Spoel et al. 2005). AMBERFF14SB force field and general AMBER force field (GAFF) were used to generate the ligand parameter, topology and restraint and the protein, respectively. The Mpro-ligand complexes were solvated in a cubic box with 1.0 nm from protein to box edge and filled with a single point charge (SPC) water model. The solvated Mpro-ligand complexes were minimized for 5000 steps. During water and ions coupling, the heavy atoms of protein and ligand were restrained. The whole system was equilibrated in two phases of 50ps NVT (constant number of particles, volume, and temperature) at 300 K followed by 1 ns NPT (constant number of particles, pressure, and temperature) ensemble at 300 K. Production stages were done over simulation times of 20 and 100 ns with NPT ensemble was adopted. Parrinello-Rahman algorithm maintains constant pressure at 1 bar and V-rescale thermostat algorithm for temperature coupling at 300 K. Long-range electrostatics under periodic conditions with a direct space cut-off 12 Å were restrained by Particle Mesh Ewald (PME) method. Time step was set to 2 fs. Coordinates and output values were collected every 10 ps.

GROMACS MD simulation toolkits were used in trajectory analysis. The g_rms and g_rmsf were used to calculate the root mean square deviation (RMSD) of starting structure to the end of simulation time and per-residue root mean square fluctuation (RMSF) of protein residues.

The Molecular mechanics-generalized Born surface area (MM-GBSA) was calculated by the g_mmpbsa tool.

### Results

Virtual screening and docking comprised a two-step process. First, an initial standard-precision (SP) docking protocol was performed, with compounds having a docking score of -8.00 or higher (453 compounds) selected for extra-precision (XP) evaluation. After SP-docking, the selected compounds were exported in SDF format and redocked using the XP-docking module. The top 14 compounds with the highest docking scores were used in MD simulations, taking lopinavir as a reference inhibitor (Tables 2&3). All of the top compounds showed favorable profiles and negative scores for Hbond, hydrophobic interactions, vdw, and coulombic interactions. Likewise, the calculated binding energy scores (MM-GBSA) were favorable and indicated strong binding profiles, with values ranging from -56.67 to -106.64 kcal/mol (Table 1).

TABLE 2

Virtual Screening & Docking Output of the Top Fourteen Compounds

| Title | Docking Score | Glide Ligand Efficiency | Glide lipo | Glide hbond |
|---|---|---|---|---|
| Rutin | -11.78 | -0.27 | -2.62 | -0.16 |
| (-)-Epigallocatechin | -11.57 | -0.35 | -2.91 | -0.65 |
| Sennoside A | -10.77 | -0.17 | -2.13 | -0.14 |
| asinex8472 | -9.83 | -0.32 | -2.83 | -1.23 |
| Atazanavir | -9.81 | -0.34 | -3.11 | -1.28 |
| asinex8485 | -9.78 | -0.33 | -2.69 | -1.33 |
| asinex6886 | -9.71 | -0.30 | -3.50 | -0.84 |
| Alpha-Mangostin | -9.14 | -0.31 | -3.68 | -0.83 |
| Glycitin | -8.83 | -0.28 | -2.96 | -0.32 |
| Birinapant | -8.81 | -0.15 | -3.67 | -0.46 |
| F2583-0433 | -8.80 | -0.29 | -2.97 | -1.20 |
| F3234-0818 | -8.65 | -0.30 | -3.03 | -0.90 |
| Lopinavir | -8.68 | -0.15 | -4.90 | -0.16 |
| Cobicistat | -8.55 | -0.10 | -4.48 | -0.26 |

TABLE 3

Virtual Screening & Docking Output of the Top Fourteen Compounds (Cont’d)

| Title | Glide evdw | Glide ecoul | Glide energy | MMGBSA dG Bind |
|---|---|---|---|---|
| Rutin | -47.64 | -27.84 | -75.49 | -88.91 |
| (-)-Epigallocatechin | -34.90 | -24.05 | -58.95 | -70.18 |
| Sennoside A | -39.11 | -18.62 | -57.73 | -61.40 |
| asinex8472 | -38.99 | -11.59 | -50.58 | -64.51 |
| Atazatiavir | -37.43 | -13.69 | -51.11 | -74.66 |
| asinex8485 | -42.63 | -11.63 | -54.25 | -73.40 |
| asinex6886 | -45.42 | -8.69 | -54.11 | -56.67 |
| Alpha-Mangostin | -42.12 | -8.79 | -50.91 | -93.46 |
| Glycitin | -32.44 | -15.39 | -47.83 | -77.38 |
| Birinapant | -60.32 | -14.06 | -74.38 | -106.64 |
| F2583-0433 | -42.87 | -17.08 | -59.95 | -89.30 |
| F3234-0818 | -43.91 | -12.30 | -56.20 | -72.79 |
| Lopinavir | -61.64 | -7.72 | -69.36 | -84.25 |
| Cobicistat | -54.69 | -14.25 | -68.94 | -82.33 |

Statistical analysis comprised determining the correlation between the obtained docking score and ligand efficiency, lipo, Hbond, vdw, coulombic, glide energy, and binding energy scores (Table 4). A strong negative correlation was observed between docking score and lipophilic interactions (r=-0.60, p>0.05), and a positive correlation with columbic interactions (r=0.74, p>0.05). This implies a predominance of electrostatic interactions in compounds binding with SARS CoV-2 Mpro.

TABLE 4

Correlation Statistics of Docking Score and Outputs of XP-Docking

| Docking Score vs.: | Glide Ligand Efficiency | Glide Lipo | Glide Hbond | Glide evdw | Glide ecoul | Glide Energy | MMGBSA dG Bind, |
|---|---|---|---|---|---|---|---|
| Pearson r | 0.3446 | -0.6011 | -0.1216 | -0.3886 | 0.741 | 0.08881 | -0.3251 |
| 95% Confidence Interval | -0.2275 to 0.7399 | -0.858 to -0.1035 | -0.6127 to 0.4371 | -0.7621 to 0.1788 | 0.3468 to 0.9127 | -0.4636 to 0.5915 | -0.7298 to 0.2483 |
| R Squared | 0.1188 | 0.3613 | 0.0148 | 0.151 | 0.5491 | 0.00788 8 | 0.1057 |
| P Value P (two- tailed) | 0.2275 | 0.0230 | 0.6787 | 0.1697 | 0.0024 | 0.7627 | 0.2567 |
| P Value Summary | Ns | * | Ns | ns | ** | ns | ns |
| Significant ? (Alpha = 0.05) | No | Yes | No | No | Yes | No | No |
| Number XY Pairs | 14 | 14 | 14 | 14 | 14 | 14 | 14 |

The determined binding features for each compound with Mpro are provided in FIGS. 1A-1F. The binding site is mostly composed of hydrophobic residues (THR24, THR25, LEU27, VAL42, MET49, PRO52, TYR54, PHE140, LEU141, MET165, LEU167, and THR190); also present are few positively charged residues (ARG188), negatively charged residues (GLU166 and ASP187), and neutral residues (CYS44, SER 144, GLN189, and GLN192).

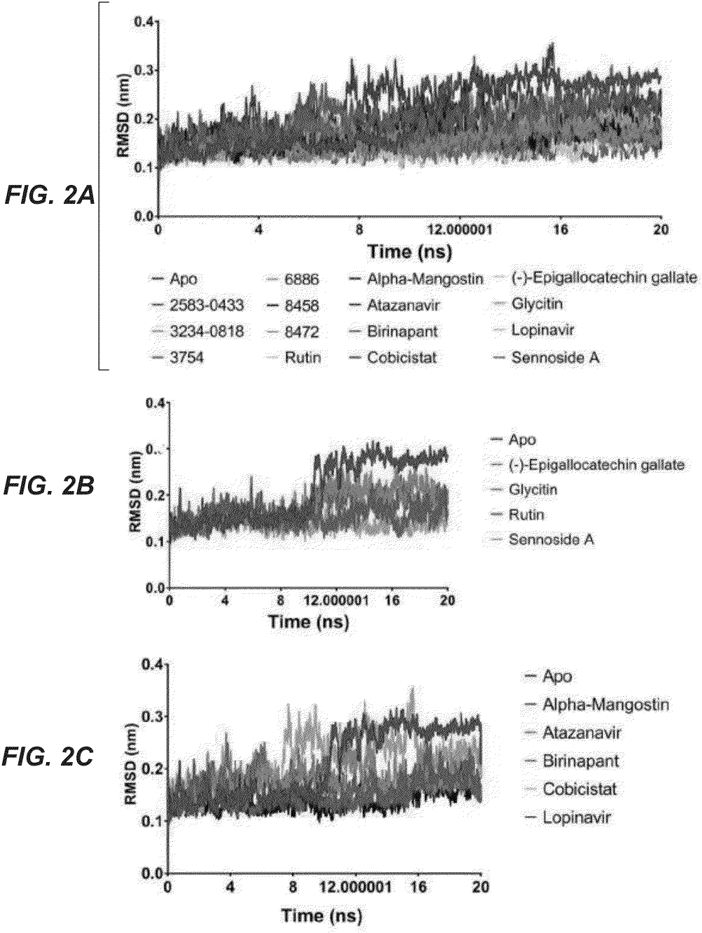

The top-ranked compounds from XP-docking were subjected to MD simulation followed by post-dynamic binding energy analysis. Compound filtering in two steps was used. First, all 14 compounds were simulated in MD for 20 ns and their RMSD, RMSF, and MM-GBSA values were calculated. The top four compounds were then examined in a more detailed 100 ns simulation for the second stage. After 20 ns simulations, structural changes in Mpro backbone residues relative to the initial structure (RMSD) were compared (FIGS. 2A-2C). Except for Apo Mpro and Mpro coupled with cobicistat and glycitin, all therapeutic complexes demonstrated high stability.

The MM-GBSA binding energies of the 14 compounds ranged from -42.627 kcal/mol to -42.627 kcal/mol. The top six compounds showed MM-GBSA binding energies ranging from -102.564 to -139.154, indicating a likely substantial binding affinity. Furthermore, all of the investigated compounds had low structural RMSD throughout the 20 ns simulations, with RMSD values as low as 0.21 nm (Table 5).

TABLE 5

MM-GBSA Binding Energy and Average Structure RMSD of Top 14 Compounds After MDS for 20 ns

| Compound ID | Binding Energy (kcal/mol) | Average Structure RMSD (nm) |
|---|---|---|
| Birinapant | -139.154 | 0.171 |
| Atazanavir | -130.299 | 0.180 |
| Lopinavir | -114.654 | 0.138 |
| Cobicistat | -111.296 | 0.214 |
| Alpha--Mangostin | -107.446 | 0.151 |
| 8472 | -102.564 | 0.151 |
| (-)-Epigallocatechin gallate | -88.348 | 0.155 |
| 3754 | -83.21 | 0.160 |
| 3234-0818 | -77.978 | 0.157 |
| 2583-0433 | -73.954 | 0.201 |
| 8458 | -70.288 | 0.165 |
| 6886 | -70.18 | 0.171 |
| Rutin | -47.388 | 0.169 |
| Glycitin | -42.627 | 0.175 |
| Sennoside A | 59.744 | 0.138 |

In order to obtain deeper insight into the strongest-binding drugs, the four drugs with binding energy >-100 kcal/mol (alpha-mangostin, atazanavir, birinapant, and lopinavir) were further subjected to 100 ns MD simulations followed by analysis of binding free energy, RMSD, RMSF, hydrogen bond length, and radius of gyration. All four had promising binding free energy values (Table 4). Specifically, the estimated MM-GBSA binding energy values were -117.90, -117.83, -121.80, and -112.80 for alpha-mangostin, atazanavir, birinapant, and lopinavir, respectively. The three drugs alpha-mangostin, atazanavir, and birinapant are implied by these values to have superior binding over lopinavir.

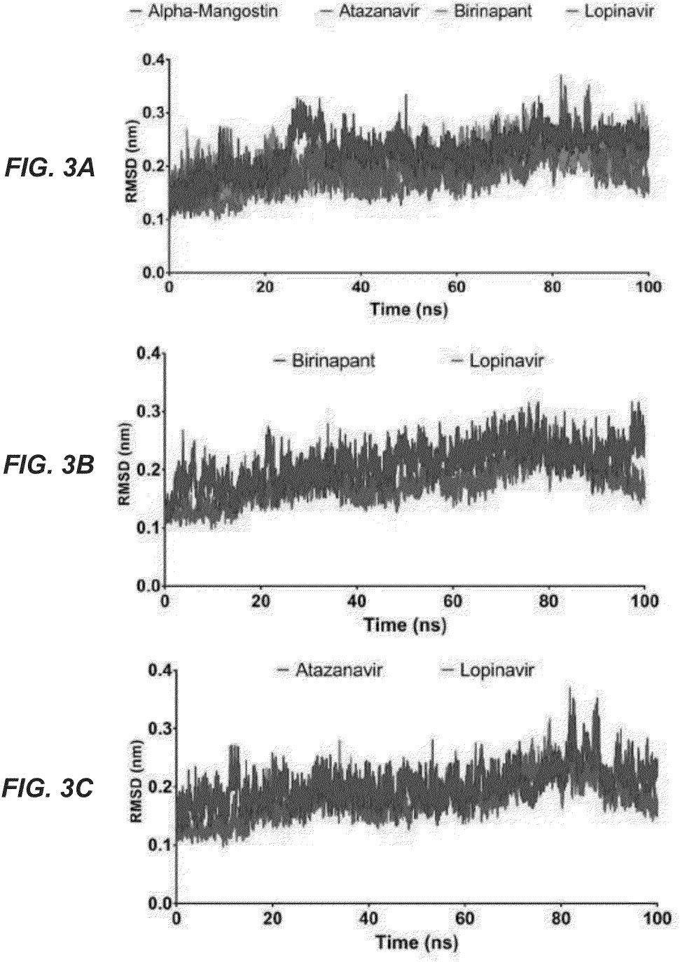

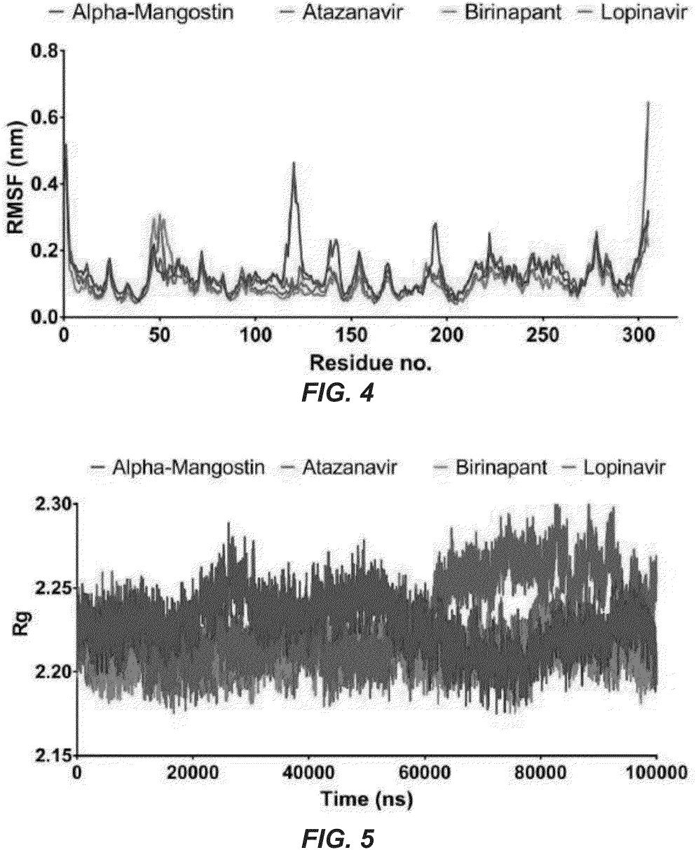

After 100 ns MDs, average RMSD values of 0.23, 0.20, 0.21, and 0.18 nm were obtained for alpha-mangostin, atazanavir, birinapant, and lopinavir, respectively. Relative to experimental IZN/ISD ranges, these values indicate marked stability of all four drugs when complexed with Mpro. Such complexes can be ranked in terms of stability as follows: lopinavir>atazanavir>birinapant>alpha-mangostin. The low ranking of alpha-mangostin can be explained by the abrupt drift in its RMSD value at 22 nm, observable in FIGS. 3A-3C. The energy value obtained for alpha-mangostin likewise indicates a lower affinity to Mpro. Meanwhile, the per-residue RMSF (FIG. 4) shows conserved RMSF features in Mpro complexes with lopinavir, birinapant, and atazanavir. Surprisingly, alpha-mangostin showed several protein fragments with very high RMSD values of 0.4 nm. Nonetheless, based on observations of binding energy, RMSD, and RMSF values, we can exclude alpha-mangostin from being repurposed on the basis of interaction with SARS CoV-2 Mpro.

Radius of gyration can be taken as a measure of the compactness of a system, with high Rg values indicating lower compactness or more unfolded protein, while low Rg indicates more stable structures. All four top drugs had an average Rg value of 2.21 nm; these similar Rg values indicate the stability of the examined drugs when complexed with Mpro. FIG. 5 shows the variation in Rg obtained during 100 ns MD simulations. Birinapant and ritonavir showed almost similar profiles with less-variable Rg, while alpha-mangostin and atazanavir showed biphasic profiles of alternating higher and lower Rg. Nonetheless, the overall average Rg values were similar for the four drugs.

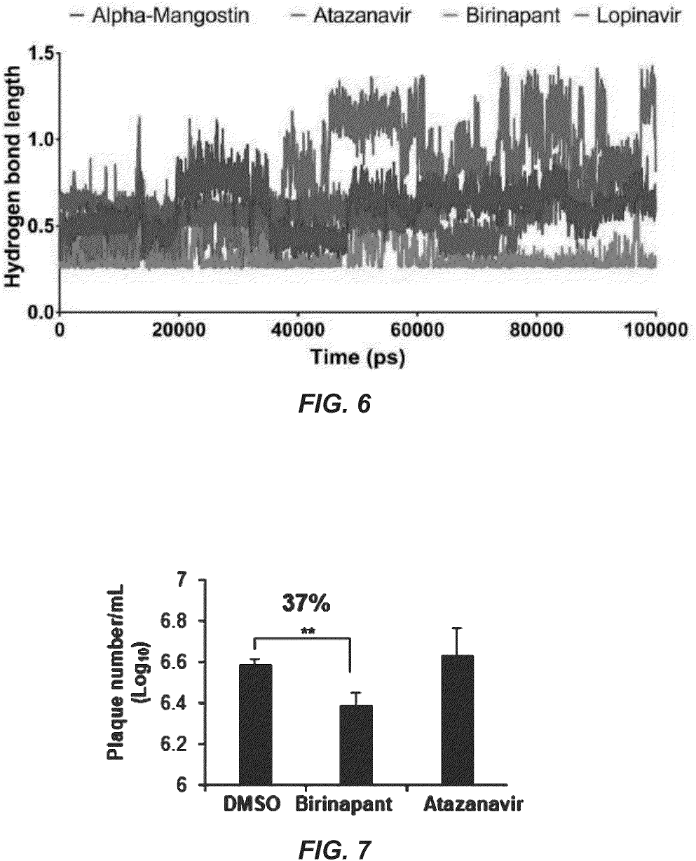

FIG. 6 shows the average hydrogen bond length obtained using GLU 166 over a 100 ns simulation. Birinapant, with an average length of 0.25 nm, demonstrated the only stable binding with GLU166.

The dominant interactions during drug recognition by Mpro were analyzed through post-dynamic energy decomposition analysis (Table 5). The results revealed that vdw and electrostatic interactions were the predominant forces for all four drugs when binding to Mpro. More specifically, vdw was the major force for alpha-mangostin, atazanavir, and lopinavir, while electrostatic forces were the major contributor for birinapant binding, with lesser contribution from vdw.

### Example 2

### In Vitro SARS-CoV-2 Plaque Inhibition Assay

African green monkey kidney Vero E6 cells were purchased from the Korean Cell Line Bank (Seoul, Korea). The cells were incubated in 95% air and 5% CO2 at 37° C. in Dulbecco’s modified Eagle’s medium (DMEM, Thermo Fisher Scientific, Waltham, MA, USA) containing 10% fetal bovine serum (FBS, Thermo Fisher Scientific), 25 mM HEPES, 100 U/mL penicillin, and 100 µg/mL streptomycin. SARS-CoV-2 (NCCP No. 43326) was provided by the National Culture Collection for Pathogens (Osong, Korea).

Vero E6 cells (5 x 10⁴ cells/well 6-well plates) were cultured overnight. The cells were infected with SARS-CoV-2 in PBS (0.1 MOI) for 1 h with shaking at 15-20 min intervals in a CO2 incubator at 37° C., and then 2 ml of DMEM containing 2% FBS was added to each well. After 3 h incubation in a CO₂ incubator at 37° C., the cells were treated with DMSO (0.1 %), birinapant (10 µM) or atazanavir (10 µM) and the plates were incubated for an additional 48 h. Supernatants of virus-infected cells were collected and virus replication was quantified using the plaque formation assay.

Correlation statistics were carried out by GraphPad Prism software. The Pearson’s correlation coefficient was used to conclude the significance of results.

### Results

Plaques inhibition assays in Vero E6 cells were used to explore the drugs inhibitory properties against SARS-CoV-2 infection. At a concentration of 10 µM, atazanavir had no antiviral effects. Birinapant, on the other hand, reduced the production of SARS-CoV-2 plaques by 37% (FIG. 7). Treatment with birinapant significantly inhibited the SARS-CoV-2 plaque formation in a dose-dependent manner. The estimated IC₅₀ value for birinapant was 18±3.6 µM.

Following validation of activity against SARS-CoV-2, the interaction of birinapant with several viral proteins, including papain-like protease, main protease, RNA-dependent RNA polymerase, and Spike RBD, was investigated. Substantial interaction with RBD was found, with a binding free energy value of -120.6, indicating strong binding. The interaction was derived by combined hydrophobic interactions and hydrogen bonds.

It is to be understood that the SARS-CoV-2 inhibitors are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of preventing SARS CoV-2 infection, comprising administering a therapeutically effective amount of a pharmaceutical composition including birinapant to a subject in need thereof.

2. The method of claim 1, wherein the pharmaceutical composition further comprises at least one additional antiviral agent.

3. The method of claim 1, wherein the pharmaceutical composition is administered by a route selected from the group consisting of orally, nasally, rectally, intracisternally, intraperitoneally, transdermally, and parenterally.

4. The method of claim 1, wherein the subject is a mammal.

5. The method of claim 4, wherein the mammal is a human being.

6. A method of treating a subject infected with SARS COV-2, comprising administering a therapeutically effective amount of a pharmaceutical composition including birinapant to the subject infected with SARS COV-2 .

7. The method of claim 6, wherein the pharmaceutical composition further comprises at least one additional antiviral agent.

8. The method of claim 6, wherein the pharmaceutical composition is administered by a route selected from the group consisting of orally, nasally, rectally, intracisternally, intraperitoneally, transdermally, and parenterally.

9. The method of claim 6, wherein the subject is a mammal.

10. The method of claim 9, wherein the mammal is a human being.
